(19) United States
(12) Patent Application Publication
(10) Pub. No.: US 2006/0257871 A1
(43) Pub. Date: Nov. 16, 2006

(54) One step real-time rt pcr kits for the universal detection of organisms in industrial products

(76) Inventors: Franck Chaubron, Chamalieres (FR); Anne Celine Martin -Minvielle, Chamalieres (FR); Sophie Groulon, Cournon D'Auvergne (FR)

Correspondence Address: WHYTE HIRSCHBOECK DUDEK S.C., ONE EAST MAIN STREET, SUITE 300, MADISON, WI, 53703-3300, US

(21) Appl. No.: 10/534,647

(22) PCT Filed: Nov. 3, 2003
(86) PCT No.: PCT/IB03/05312

§ 371 Date: Apr. 3, 2006

Related U.S. Application Data
(60) Provisional application No. 60/425,327, filed on Nov. 12, 2002.

(51) Int. Cl. C12Q 1/68

(52) U.S. Cl. 435/6

(57) ABSTRACT

This invention is related to a novel sample preparation, probes, couple primer sets for one step real time reverse transcriptase polymerise chain reaction (RT-PCR), methods and kits for the universal detection of alive bacteria and/or fungus-yeast in pharmaceutical, cosmetic and non clinical samples.

### TECHNICAL FIELD

The invention pertains to the field of methods and reagents for detecting bacteria and fungus-yeast found in pharmaceutical, cosmetic and non clinical samples.

More specifically, the present invention relates to a sample preparation, primer sets, probe sets and methods for one step real-time RT PCR kits for the universal detection of alive bacteria and fungus-yeast in sterile or non sterile industrial product in less than 24 hours.

### BACKGROUND OF THE INVENTION

The use of specific polynucleotide sequences or Peptide Nucleic Acid as primers and/or probes for the recognition of contaminant and infectious agents is becoming a valuable alternative to problematic growth requirements assays, visible (colony) growth features, microscopic morphology, staining reactions, and biochemical characteristics. Most of the time these technologies are too slow for a real used in industrial controls.

For example, PCT publication WO84/02721; published Jul. 19, 1984 describes the use of nucleic acid probes complementary to targeted nucleic acid sequences composed of ribosomal RNA, transfer RNA, or other RNA in hybridization procedures to detect the target nucleic acid sequence. While the assay may provide greater sensitivity and specificity than known DNA hybridization assays, hybridization procedures which require the use of a complementary probe are generally dependent upon the cultivation of a test organism and are, therefore, unsuitable for rapid analysis.

These probes are useful in hybridizing to RNA amplified by the Reverse Transcriptase Polymerase Chain Reaction (RT PCR). RT-PCR is a powerful ribonucleic acid amplification technique that can be used for the detection of small numbers of ribonucleotide acid targets from bacteria and/or from fungus-yeast whose in vitro cultivation is difficult or lengthy. RT PCR requires the presence of living specimens for detection. In its simplest form, RT PCR is an in vitro method for the enzymatic synthesis of specific cDNA sequences. Using one oligonucleotide primers that hybridize to RNA strand and flank the region of interest in the target cDNA, several cDNA are synthetised by Reverse Transcriptase. A repetitive series of cycles involving template denaturation, primer annealing, and extension of annealed primers by DNA polymerase results in the exponential accumulation of a specific fragment whose termini are defined by the 5′ ends of the primers. PCR produce a selective enrichment of a specific DNA sequence by a factor of 10.sup.12. The PCR method is described in Saiki et al, 1985, Science 230:1350 and is the subject of U.S. Pat. Nos. 4,683,195, 4,683,202 and 4,800,159 (these references are incorporated herein by reference). This method has been used to detect the presence of the aberrant sequence in the beta-globin gene which is related to sickle cell anemia (Saiki et al., 1985, supra) and the human immunodeficiency virus (HIV) RNA (Byrne et al., 1988, Nuc. Acids Res. 16:4165).

In order to successfully treat a contamination caused by a bacteria or fungus-yeast in a sterile or non sterile product from industry, a rapid and accurate detection is required. Bacterial and fungus-yeast detection have traditionally been accomplished by pure culture isolation, followed by identification procedures that make use of knowledge of specimen source, growth requirements, visible (colony) growth features, microscopic morphology, staining reactions, and biochemical characteristics.

It is clear that a rapid diagnostic method, less than 24 hours, for detecting bacteria and fungus-yeast in industrial samples with the same sensitivity as culture would be a significant improvement over currently used methods.

### SUMMARY OF THE INVENTION

The present invention pertains to methods and reagents for the rapid detection of bacteria and fungus-yeast in sterile and non sterile product in less than 24 hours.

In a preferred embodiment, a target region from a one-step Reverse Transcriptase Polymerase Chain Reaction of RNA and the resultant amplified DNA is treated with probes which can hybridize to the amplified DNA of bacteria or fungus-yeast but not other organisms (mammalian, plant, insects . . . ) or virus.

The Tth DNA polymerase is a thermostable enzyme with RNA-dependent Reverse Transcriptase activity and with DNA-dependent Polymerase activity, allowing the combination of RT and PCR in a single-tube reaction resulting in a faster analysis of presence of RNA from bacteria, fungus-yeast.

Using one-step Real-time Reverse Transcriptase Polymerase Chain Reaction, the invention enable the user to perform a rapid RT-PCR and simultaneously detect and quantify the presence of RNA from bacteria and/or fungus-yeast by monitoring fluorescence during real time polymerase chain reaction amplification with any risk of false positive due to opening tube between RT and PCR and from possible PCR product environmental contamination due to precedent amplification reactions in the laboratory.

### DETAILED DESCRIPTION OF THE INVENTION

The methods of the present invention thus enable determination of the presence of bacteria and/or fungus-yeast more rapidly than technologies with prior art detection methods.

Using one-step Real-time Reverse Transcriptase Polymerase Chain Reaction, the invention enable the user to perform a rapid RT-PCR and simultaneously analyse and quantify the presence of RNA from bacteria and/or fungus-yeast by monitoring fluorescence during real time polymerase chain reaction amplification with any risk of false positive due to opening tube between RT and PCR and from possible PCR product environmental contamination due to precedent amplification reactions.

The basic RT PCR process is carried out as follows.

A sample is provided which needs to be tested or which is suspected of contain a particular ribonucleic acid sequence of interest, the “target sequence.” The ribonucleic acid contained in the sample may be first reverse transcribed into cDNA (using enzyme like Tth DNA polymerase as purified enzyme and a oligonucleotide or PNA), and then denatured, using physical means, which are known to those of skill in the art. A preferred physical means for strand separation involves heating the nucleic acid until it is completely (>99%) denatured. Methods for the amplification of RNA targets using a thermostable DNA polymerase are described in PCT/US90/07641, filed Dec. 21, 1990, and incorporated herein by reference.

The denatured DNA strands are then incubated in the same tube with the selected oligonucleotide primers under hybridization conditions, conditions which enable the binding of the primers to the single DNA strands. As known in the art, the primers are selected so that their relative positions along a duplex sequence are such that an extension product synthesized from one primer, when it is separated from its complement, serves as a template for the extension of the other primer to yield a replicate chain of defined length.

The primer must be sufficiently long to prime the synthesis of extension products in the presence of the agent for polymerization. The exact length of the primers will depend on many factors, including temperature, source of the primer and use of the method.

Preferred oligonucleotide primers for use in the present invention are selected from the group consisting of

| | |
|---|---|
| Seq ID No 1 | |
| TGGAGCATGTGGTTTAATTCGA | [primer forward] |
| Seq ID No 2 | |
| TGCGGGACTTAACCCAACA | [primer reverse] |
| Seq ID No 3 | |
| AGAGTTTGATCATGGCTCAGA | [primer forward] |
| Seq ID No 4 | |
| TTACCCCACCTACTAGCTAAT | [primer reverse] |
| Seq ID No 5 | |
| GYGGAGCATGTGGYTTAATTCG | [primer forward] |
| Seq ID No 6 | |
| TTGCGCTCGTTRCGGGACTT | [primer reverse] |
| Seq ID No 7 | |
| GGGAAACTCACCAGGTCCA | [primer forward] |
| Seq ID No 8 | |
| CGTTATCGCAATTAAGCAGACA | [primer reverse] |
| Seq ID No 9 | |
| GGTAACGGGGAATWAGGGTTC | [primer forward] |
| Seq ID No 10 | |
| TTGGGTAATTTGCGCGCCTG | [primer reverse] |

Universal code: Y = (C/T), R = (A/G), W = (A/T)

Template-dependent extension of the oligonucleotide primer(s) is then catalyzed by the polymerizing agent (in the presence of adequate amounts of the four deoxyribonucleoside triphosphates (DATP, dGTP, dCTP, and dTTP) or analogs), in a reaction medium which is comprised of the appropriate salts, metal cations, and pH buffering system.

The products of the synthesis are duplex molecules consisting of the template strands and the primer extension strands, which include the target sequence. These products, in turn, serve as templates for another round of replication. In the second round of replication, the primer extension strand of the first cycle is annealed with its complementary primer; synthesis yields a “short” product which is bounded on both the 5′- and the 3′-ends by primer sequences or their complements. Repeated cycles of denaturation, primer annealing, and extension result in the exponential accumulation of the target region defined by the primers. Sufficient cycles are run to achieve the desired amount of polynucleotide containing the target region of nucleic acid. The desired amount may vary, and is determined by the function which the product polynucleotide is to serve.

The PCR method is performed in a fashion where all of the reagents are added simultaneously, in one step.

In a preferred method, the RT PCR reaction is carried out as an automated process which utilizes a thermostable enzyme like Tth.

The types of machines used are commercially available from Roche Diagnostics (LigthCycler), Cepheid (Smart Cycler, GeneXpert), BioRad (Icycler), Corbett Research (Rotor-Gene) . . . and most suitable equipment developed for real time PCR assays and commercial use.

Those skilled in the art will also be aware of the problems of contamination of a PCR by the nucleic acid from bacteria previously present in water used for buffer and resulting in non specific amplification. Methods to reduce these problems are provided by using adequate filtration systems to avoid DNA strand fragments with a size higher than 100 bp. All reagents used in the RT PCR reaction have to be processed before using.

During amplification by PCR, the target polynucleotides may be detected directly by hybridization with a probe polynucleotide which forms a stable hybrid with the target sequence under high stringency to low stringency hybridization and washing conditions.

Probes are typically labeled with non-radioactive labeling systems, such as fluoresceins and derivated systems.

Therefore, in one embodiment, the invention relates to a method and kit for determining the presence of bacteria or fungus-yeast ribonucleic acid (RNA) in a sample suspected of containing said bacteria and/or fungus, wherein said polynucleotide comprises a selected target region, said method comprising:

(a) extract bacteria or fungus-yeast ribonucleic acid (RNA) from the sample up to 1000 ml by centrifiltration on membranes and/or DEAE resin following by incubation with DNAse.

(b) incubating the bacteria or fungus-yeast ribonucleic acid (RNA) with a thermostable enzyme with RNA-dependent Reverse Transcriptase activity and with DNA-dependent Polymerase activity, allowing the combination of RT and PCR in a single-tube reaction, such as Tth DNA polymerase or an enzyme like Tth DNA polymerase, and polynucleotide primers with a nucleotide sequence selected from the group consisting of

| | |
|---|---|
| Seq ID No 2 | |
| TGCGGGACTTAACCCAACA | [primer reverse] |
| Seq ID No 4 | |
| TTAACCCCAACCTACTAGCTAAT | [primer reverse] |
| Seq ID No 6 | |
| TTGCGCTCGTTRCGGGACTT | [primer reverse] |
| Seq ID No 8 | |
| CGTTATCGCAATTAAGCAGACA | [primer reverse] |
| Seq ID No 10 | |
| TTGGGTAATTTGCGCGCCTG | [primer reverse] |

under conditions which allow hybridization of the polynucleotide to the ribonucleotide target region and Reverse Transcriptase activity of the said polymerase, or enzyme like Tth, for cDNA synthesis; and

(c) amplified the cDNAs formed to a detectable level by Polymerase Chain Reaction with said polymerase enzyme like Tth DNA polymerase and polynucleotide primers and probes with a nucleotide sequence selected from the group consisting of

| | |
|---|---|
| Seq ID No 1 | |
| TGGAGCATGTGGTTTAATTCGA | [primer forward] |
| Seq ID No 2 | |
| TGCGGGACTTAACCCAACA | [primer reverse] |
| Seq ID No 3 | |
| AGAGTTTGATCATGGCTCAGA | [primer forward] |
| Seq ID No 4 | |
| TTACCCCACCTACTAGCTAAT | [primer reverse] |
| Seq ID No 5 | |
| GYGGAGCATGTGGYTTAATTCG | [primer forward] |
| Seq ID No 6 | |
| TTGCGCTCGTTRCGGGACTT | [primer reverse] |
| Seq ID No 7 | |
| GGGAAACTCACCAGGTCCA | [primer forward] |
| Seq ID No 8 | |
| CGTTATCGCAATTAAGCAGACA | [primer reverse] |
| Seq ID No 9 | |
| GGTAACGGGGAATWAGGGTTC | [primer forward] |
| Seq ID No 10 | |
| TTGGGTAATTTGCGCGCCTG | [primer reverse] |
| Seq ID No 11 | |
| TGCATGGYTGTCGTCAGCTCGTG | [probe forward] |
| Seq ID No 12 | |
| GAGTGGCGGACGGGTGAGTAA | [probe forward] |
| Seq ID No 13 | |
| ACAGGTGGTGCATGGTTGTC | [probe forward] |
| Seq ID No 14 | |
| TCAGCTCGTGTCGTGAGATGTT | [probe forward] |
| Seq ID No 15 | |
| ACGGTGCTGCATGGCTGTC | [probe forward] |
| Seq ID No 16 | |
| TCAGCTCGTGTTGTGAAATGTT | [probe forward] |
| Seq ID No 17 | |
| AGGATTGACAGATTGAGAGCTCTT | [probe forward] |
| Seq ID No 18 | |
| CGGAGAGGGAGCCTGAGAA | [probe forward] |
| Seq ID No 19 | |
| CGGCTACCACATCCAAGGAA | [probe forward] |

The cDNA target sequence can be synthetised by Reverse Transcriptase activity of Tth or an enzyme like Tth and is amplified by the DNA-dependent Polymerase activity of the DNA polymerase in the same tube by means of one step real time RT-PCR.

More particularly, the composition for detecting bacteria comprises a polynucleotide primers and a probe consisting of the sequence

| | |
|---|---|
| Seq ID No 1 | |
| TGGAGCATGTGGTTTAATTCGA | [primer forward] |
| Seq ID No 2 | |
| TGCGGGACTTAACCCAACA | [primer reverse] |
| Seq ID No 11 | |
| TGCATGGYTGTCGTCAGCTCGTG | [probe forward] |

Alternatively, the composition for detecting bacteria comprises a polynucleotide primers and a probe consisting of the sequence

| | | |
|---|---|---|
| Seq ID No 3 | AGAGTTTGATCATGGCTCAGA | [primer forward] |
| Seq ID No 4 | TTACCCCACCTACTAGCTAAT | [primer reverse] |
| Seq ID No 12 | GAGTGGCGGACGGGTGAGTAA | [probe forward] |

The invention may also be practiced with a composition for detecting bacteria which comprises a polynucleotide primers and a probe consisting of the sequence

| | |
|---|---|
| Seq ID No 5 | |
| GYGGAGCATGTGGYTTAATTCG | [primer forward] |
| Seq ID No 6 | |
| TTGCGCTCGTTRCGGGACTT | [primer reverse] |
| Seq ID No 13 | |
| ACAGGTGGTGCATGGTTGTC | [probe forward] |
| Seq ID No 14 | |
| TCAGCTCGTGTCGTGAGATGTT | [probe forward] |
| Seq ID No 15 | |
| ACAGGTGCTGCATGGCTGTC | [probe forward] |
| Seq ID No 16 | |
| TCAGCTCGTGTTGTGAAATGTT | [probe forward] |

The invention also concerns the above mentioned method and kit wherein the composition for detecting fungus-yeast comprises a polynucleotide primers and a probe consisting of the sequence

| | |
|---|---|
| Seq ID No 7 | |
| GGGAAACTCACCAGGTCCA | [primer forward] |
| Seq ID No 8 | |
| CGTTATCGCAATTAAGCAGACA | [primer reverse] |
| Seq ID No 17 | |
| AGGATTGACAGATTGAGAGCTCTT | [probe forward] |

Alternatively, the composition for detecting fungus-yeast comprises a polynucleotide primers and a probe consisting of the sequence

| | | |
|---|---|---|
| Seq ID No 9 | GGTAACGGGGAATWAGGGTTC | [primer forward] |
| Seq ID No 10 | TTGGGTAATTTGCGCGCCTG | [primer reverse] |
| Seq ID No 18 | CGGAGAGGGAGCCTGAGAA | [probe forward] |
| Seq ID No 19 | CGGCTACCACATCCAAGGAA | [probe forward] |

The preferred combination of primers and probes used for detection all bacteria and/or fungus-yeast consists of the sequence:
Seq ID No 1+Seq ID No 2+Seq ID No 11
   or
Seq ID No 3+Seq ID No 4+Seq ID No 12
   or
Seq ID No 5+Seq ID No 6+Seq ID No 13+Seq ID No 14+Seq ID No 15+Seq ID No 16
   or
Seq ID No 7+Seq D No 8+Seq ID No 17
   or
Seq ID No 9+Seq ID No 10+Seq ID No 18+Seq ID No 19
   or
Seq ID No 1+Seq ID No 2+Seq ID No 11+Seq ID No 7+Seq ID No 8+Seq ID No 17
   or
Seq ID No 3+Seq D No 4+Seq ID No 12+Seq ID No 7+Seq ID No 8+Seq ID No 17
   or
Seq ID No 5+Seq ID No 6+Seq ID No 13+Seq ID No 14+Seq ID No 15+Seq ID No 16+Seq ID No 9+Seq ID No 10+Seq ID No 18+Seq ID No 19

As mentioned above, the polynucleotide primers and probes may be natural nucleic acid or Peptide Nucleic Acid (PNA) which can hybridize to nucleic acid (DNA and RNA).

The RNA may also be quantified and compared with quantified external standard RNA from by exemple Escherichia coli and Candida spp.

By way of further specificity, the following probe nucleotide base pair data is provided. Preferred oligonucleotide probes for use in the present invention are selected from the group consisting of

| | |
|---|---|
| Seq ID No 11 | |
| TGCATGGYTGTCGTCAGCTCGTG | [probe forward] |
| Seq ID No 12 | |
| GAGTGGCGGACGGGTGAGTAA | [probe forward] |
| Seq ID No 13 | |
| ACAGGTGGTGCATGGTTGTC | [probe forward] |
| Seq ID No 14 | |
| TCAGCTCGTGTCGTGAGATGTT | [probe forward] |
| Seq ID No 15 | |
| ACAGGTGCTGCATGGCTGTC | [probe forward] |
| Seq ID No 16 | |
| TCAGCTCGTGTTGTGAAATGTT | [probe forward] |
| Seq ID No 17 | |
| AGGATTGACAGATTGAGAGCTCTT | [probe forward] |
| Seq ID No 18 | |
| CGGAGAGGGAGCCTGAGAA | [probe forward] |
| Seq ID No 19 | |
| CGGCTACCACATCCAAGGAA | [probe forward] |

Reverse probes are not usable because in a one-step RT-PCR the probe should not hybridize the RNA sequence zone where the cDNA is synthetised by Reverse Transcriptase.

The sequence of the preferred oligonucleotide primers and probes of the invention are based on the rRNA gene. Oligonucleotide rRNA gene for the detection of nucleic acids from various microorganisms have been described in the scientific literature. For example, universal bacterial probes have been described by Wilson et al., 1990, J. Clinical Microbiology 28:1942-1946, and Chem et al., 1989, FEMS Microbiology Letters 57:19-24.

Examples of genus- and species-specific probes have been described by Barry et al., 1990, Biotechnology 8:233-236, Atlas and Bej, “PCR protocols: A guide to method and application,” p. 399-406; and in Gen-probe international patent application WO88/03957 (these references are incorporated herein by reference). The invention claimed in this application differs from these inventions in the range of target detected (all bacteria-fungus-yeast) and the application focus (not clinical).

The use of a panel of rRNA probes, including a universal bacterial probe, gram-positive and gram-negative probes and species or group specific probes provides clinically useful information, not a single universal bacterial probe; since different pathologies, drugs and antibiotic therapy is recommended for various bacterial—fungus-yeast infections.

In these precedent patents, universal bacterial probes are using only for positive controls. Universal primers for bacterial-fungus-yeast are using for identification after cloning and sequencing of the amplified product or hybridization on a DNA chip. The used of rRNA targets for sterility controls for detection of alive bacteria and fungus-yeast in sterile or non sterile industrial product has not been described before this invention.

Preferred universal couple of primers for the one step RT PCR detection of bacteria and fungus-yeast comprise a probing nucleobase sequence selected from the group consisting of

| | |
|---|---|
| Seq ID No 1 | |
| TGGAGCATGTGGTTTAATTCGA | [primer forward] |
| Seq ID No 2 | |
| TGCGGGACTTAACCCAACA | [primer reverse] |
| Seq ID No 3 | |
| AGAGTTTGATCATGGCTCAGA | [primer forward] |
| Seq ID No 4 | |
| TTACCCCACCTACTAGCTAAT | [primer reverse] |
| Seq ID No 5 | |
| GYGGAGCATGTGGYTTAATTCG | [primer forward] |
| Seq ID No 6 | |
| TTGCGCTCGTTRCGGGACTT | [primer reverse] |
| Seq ID No 7 | |
| GGGAAACTCACCAGGTCCA | [primer forward] |
| Seq ID No 8 | |
| CGTTATCGCAATTAAGCAGACA | [primer reverse] |
| Seq ID No 9 | |
| GGTAACGGGGAATWAGGGTTC | [primer forward] |
| Seq ID No 10 | |
| TTGGGTAATTTGCGCGCCTG | [primer reverse] |

Preferred universal probes for the detection of bacteria and fungus-yeast comprise a probing nucleobase sequence selected from the group consisting of

| | |
|---|---|
| Seq ID No 11 | |
| TGCATGGYTGTCGTCAGCTCGTG | [probe forward] |
| Seq ID No 12 | |
| GAGTGGCGGACGGGTGAGTAA | [probe forward] |
| Seq ID No 13 | |
| ACAGGTGGTGCATGGTTGTC | [probe forward] |
| Seq ID No 14 | |
| TCAGCTCGTGTCGTGAGATGTT | [probe forward] |
| Seq ID No 15 | |
| ACAGGTGCTGCATGGCTGTC | [probe forward] |
| Seq ID No 16 | |
| TCAGCTCGTGTTGTGAAATGTT | [probe forward] |
| Seq ID No 17 | |
| AGGATTGACAGATTGAGAGCTCTT | [probe forward] |
| Seq ID No 18 | |
| CGGAGAGGGAGCCTGAGAA | [probe forward] |
| Seq ID No 19 | |
| CGGCTACCACATCCAAGGAA | [probe forward] |

The probes and primer sets, methods and kits of this invention are particularly well suited for use in simplex or multiplex one step RT PCR assays wherein all the bacteria and/or fungus-yeast in a sample can be detected alive and quantitated. The total number of colony forming units (CFU) of bacteria and/or fungus-yeast can be directly determined.

The following examples are intended to be illustrative of the various methods and compounds of the invention.

### EXAMPLE 1

A preferred method for analysis of sample by single filtration (filterable liquids). Specificity of extraction from bacteria or fungus-yeast ribonucleotide from the sample up to 1000 mL by centrifiltration following by an incubation with DNase.

1—The liquid sample (up to 1000 mL) is passed through a polycarbonate membrane (up to 0,45 μm) or PVDF membrane (up to 0,45 μm) or PES membrane (up to 0,45 μm) via centrifugation (swing rotor) at 2000 g or a vacuum pump.

Enzymatic Lysis

2—Transfer the filter in a 50 mL sterile tube with up to 1 mL of enzymatic lysis buffer and then incubate at 35° C.±2° C. for up to one hour.

3—Centrifuge the liquid (lysis buffer) for 5 minutes at 2000 g.

Or Mecanic Lysis

2—Recovery bacteria and/or fungus-yeast in 600 μL of media. Incubate for up to one hour at 37° C.±1° C.

3—Add x mg of acid-washed glass beads (100-500 μm in diameter) and x μL of lysis buffer. Disrupt cells in the Mixer Mills MM300 (Retsch) for up to 90 minutes at maximal speed.

4—The lysat is processed for RNA purification with commercial kits. Our preferred RNA extraction kit is the “MagNaPure LC RNA isolation kit II” on the workstation MagNaPure LC™ (Roche Diagnostics). The elution volume is up to 100 μL. Incubation with DNase is processed during the purification.

5—2 μL (up to 5 μL) of pure RNA extract is used for the one step real time RT-PCR (LightCycler™) with enzyme like Tth and the following program with Taqman Probe:

| | |
|---|---|
| I: Reverse transcription | 61° C./20 min (20° C./sec) |
| II: Denaturation | 95° C./30 secondes (20° C./sec) |
| III: PCR (35 cycles) | 95° C./5 seconds (20° C./sec) |
| | 60° C./30 seconds (20° C./sec) |

The emitted fluorescence is measured at the end of the 60 seconds.

6—2 μL (up to 5 μL) of pure RNA extract is used for the one step real time RT-PCR (LightCycler™) with enzyme like Tth and the following program with Hybridization Probe:

| | |
|---|---|
| I: Reverse transcription | 61° C./20 min (20° C./sec) |
| II: Denaturation | 95° C./30 secondes (20° C./sec) |
| III: PCR (35 cycles) | 95° C./2 seconds (20° C./sec) |
| | 58° C./8 seconds (20° C./sec) |
| | 72° C./16 secondes (20° C./sec) |

The emitted fluorescence is measured at the end of the 8 seconds.

### EXAMPLE 2

A preferred method for analysis of sample by centrifugation (non filterable liquids). Specificity of extraction from bacteria or fungus-yeast ribonucleotide from the sample up to 1000 mL by centrifugation.

Enzymatic Lysis
1—Centrifuge the sample for 5 minutes at 11000 g, discard the supernatant.
2—Resuspend pellet in x μL of lysis buffer. Vortex and incubate for up to one hour at 37° C.±1° C.
3—Add x μL of lysis buffer. Vortex and incubate for up to 30 minutes at 50° C.+2° C.

Or Mecanic Lysis
1—Centrifuge the sample for 5 minutes at 11000 g, discard the supernatant.
2—Resuspend pellet in x μL of lysis buffer.
3—Add x mg of acid-washed glass beads (100-500 μm in diameter). Disrupt cells in the Mixer Mills MM300 (Retsch) for up to 90 minutes at maximal speed.
4—The lysat is processed for RNA purification with commercial kits. Our preferred RNA extraction kit is the “MagNaPure LC RNA isolation kit II” on the workstation MagNaPure LC™ (Roche Diagnostics). The elution volume is up to 100 μL. Incubation with DNase is processed during the purification.

5—2 μL (up to 5 μL) of pure RNA extract is used for the one step real time RT-PCR (LightCycler™) with enzyme like Tth and the following program with Taqman Probe:

| | |
|---|---|
| I: Reverse transcription | 61° C./20 min (20° C./sec) |
| II: Denaturation | 95° C./30 secondes (20° C./sec) |
| III: PCR (35 cycles) | 95° C./5 seconds (20° C./sec) |
| | 60° C./30 seconds (20° C./sec) |

The emitted fluorescence is measured at the end of the 60 seconds.

6—2 μL (up to 5 μL) of pure RNA extract is used for the one step real time RT-PCR (LightCycler™) with enzyme like Tth and the following program with Hybridization Probe:

| | |
|---|---|
| I: Reverse transcription | 61° C./20 min (20° C./sec) |
| II: Denaturation | 95° C./30 secondes (20° C./sec) |
| III: PCR (35 cycles) | 95° C./2 seconds (20° C./sec) |
| | 58° C./8 seconds (20° C./sec) |
| | 72° C./16 secondes (20° C./sec) |

The emitted fluorescence is measured at the end of the 8 seconds.

### EXAMPLE 3

A preferred method for analysis of sample by direct lysis and recovery of RNA on DiEthylAminoEthyl cellulose DEAE membrane (non filterable liquids).

Specificity of extraction from bacteria or fungus-yeast ribonucleotide from the sample up to 1000 mL by centrifugation on DEAE membrane of the lysat following by an incubation with DNase.

Enzymatic Lysis
1—Add x mL of lysis buffer in the sample. Vortex and incubate for up to one hour at 35° C.±2° C.
2—Add x mL of lysis buffer. Vortex and incubate for up to 30 minutes at 50° C.±2° C.

Or Mecanic Lysis
1—Add x mg of acid-washed glass beads (100-500 μm in diameter) and x μL of lysis buffer.
2—Disrupt cells in the Mixer Mills MM300 (Retsch) for up to 90 minutes at maximal speed.
3—After complete lysis, add the lysat on the pre-filter membrane (polypropylene 10 μm) to retain particules with a size superior at 10 μm. The liquid filtered is charged in nucleic acid.
4—Add the the lysat on the pre-wased DEAE membrane to retain all nucleic acid. Centrifuge and wash the membrane.
5—Add salt (up to 1 mL) to recovery nucleic acid in a sterile clean tube by centrifugation or with a vacuum pump.
6—The lysat is processed for RNA purification with commercial kits. Our preferred RNA extraction kit is the “MagNaPure LC RNA isolation kit II” on the workstation MagNaPure LC™ (Roche Diagnostics). The elution volume is up to 100 μL. Incubation with DNase is processed during the purification.

7—2 μL (up to 5 μL) of pure RNA extract is used for the one step real time RT-PCR (LightCycler™) with enzyme like Tth and the following program with Taqman Probe:

| | |
|---|---|
| I: Reverse transcription | 61° C./20 min (20° C./sec) |
| II: Denaturation | 95° C./30 secondes (20° C./sec) |
| III: PCR (35 cycles) | 95° C./5 seconds (20° C./sec) |
| | 60° C./30 seconds (20° C./sec) |

The emitted fluorescence is measured at the end of the 60 seconds.

8—2 μL (up to 5 μL) of pure RNA extract is used for the one step real time RT-PCR (LightCycler™) with enzyme like Tth and the following program with Hybridization Probe:

| | |
|---|---|
| I: Reverse transcription | 61° C./20 min (20° C./sec) |
| II: Denaturation | 95° C./30 secondes (20° C./sec) |
| III: PCR (35 cycles) | 95° C./2 seconds (20° C./sec) |
| | 58° C./8 seconds (20° C./sec) |
| | 72° C./16 secondes (20° C./sec) |

The emitted fluorescence is measured at the end of the 8 seconds.

1. A method and kit for determining the presence of bacteria or fungus-yeast ribonucleic acid (RNA) in a sample suspected of containing said bacteria and/or fungus, wherein said polynucleotide comprises a selected target region, said method comprising:
(a) extract bacteria or fungus-yeast ribonucleic acid (RNA) from the sample up to 1000 ml by centrifiltration on membranes and/or DEAE resin following by incubation with DNAse.
(b) incubating the bacteria or fungus-yeast ribonucleic acid (RNA) with a thermostable enzyme with RNA-dependent Reverse Transcriptase activity and with DNA-dependent Polymerase activity, allowing the combination of RT and PCR in a single-tube reaction, such as Tth DNA polymerase, and polynucleotide primers with a nucleotide sequence selected from the group consisting of

| | |
|---|---|
| Seq ID No 2 | |
| TGCGGGACTTAACCCAACA | [primer reverse] |
| Seq ID No 4 | |
| TTACCCCACCTACTAGCTAAT | [primer reverse] |
| Seq ID No 6 | |
| TTGCGCTCGTTRCGGGACTT | [primer reverse] |
| Seq ID No 8 | |
| CGTTATCGCAATTAAGCAGACA | [primer reverse] |
| Seq ID No 10 | |
| TTGGGTAATTTGCGCGCCTG | [primer reverse] |

under conditions which allow hybridization of the polynucleotide to the ribonucleotide target region and Reverse Transcriptase activity of said DNA polymerase for cDNA synthesis; and
(c) amplified the cDNAs formed to a detectable level by Polymerase Chain Reaction with said DNA polymerase and polynucleotide primers and probes with a nucleotide sequence selected from the group consisting of

| | |
|---|---|
| Seq ID No 1 | |
| TGGAGCATGTGGTTTAATTCGA | [primer forward] |
| Seq ID No 2 | |
| TGCGGGACTTAACCCAACA | [primer reverse] |
| Seq ID No 3 | |
| AGAGTTTGATCATGGCTCAGA | [primer forward] |
| Seq ID No 4 | |
| TTACCCCACCTACTAGCTAAT | [primer reverse] |
| Seq ID No 5 | |
| GYGGAGCATGTGGYTTAATTCG | [primer forward] |
| Seq ID No 6 | |
| TTGCGCTCGTTRCGGGACTT | [primer reverse] |
| Seq ID No 7 | |
| GGGAAACTCACCAGGTCCA | [primer forward] |
| Seq ID No 8 | |
| CGTTATCGCAATTAAGCAGACA | [primer reverse] |
| Seq ID No 9 | |
| GGTAACGGGGAATWAGGGTTC | [primer forward] |
| Seq ID No 10 | |
| TTGGGTTAATTTGCGCGCCTG | [primer reverse] |
| Seq ID No 11 | |
| TGCATGGYTGTCGTCAGCTCGTG | [probe forward] |
| Seq ID No 12 | |
| GAGTGGCGGACGGGTGAGTAA | [probe forward] |
| Seq ID No 13 | |
| ACAGGTGGTGCATGGTTGTC | [probe forward] |
| Seq ID No 14 | |
| TCAGCTCGTGTCGTGAGATGTT | [probe forward] |
| Seq ID No 15 | |
| ACAGGTGCTGCATGGCTGTC | [probe forward] |
| Seq ID No 16 | |
| TCAGCTCGTGTTGTGAAATGTT | [probe forward] |
| Seq ID No 17 | |
| AGGATTGACAGATTGAGAGCTCTT | [probe forward] |
| Seq ID No 18 | |
| CGGAGCGGGAGCCTGAGAA | [probe forward] |
| Seq ID No 19 | |
| CGGCTACCACATCCAAGGAA | [probe forward] |

2. The method and kit of claim 1, wherein the cDNA target sequence synthetised by Reverse Transcriptase activity of the enzyme like Tth polymerase is amplified by the DNA-dependent Polymerase activity of DNA polymerase in the same tube by means of one step real time RT-PCR.

3. The method and kit of claim 1 and 2, wherein the composition for detecting bacteria comprising a polynucleotide primers and a probe consisting of the sequence

| | |
|---|---|
| Seq ID No 1 | |
| TGGAGCATGTGGTTTAATTCGA | [primer forward] |
| Seq ID No 2 | |
| TGCGGGACTTAACCCAACA | [primer reverse] |
| Seq ID No 11 | |
| TGCATGGYTGTCGTCAGCTCGTG | [probe forward] |

4. The method and kit of claim 1 and 2, wherein the composition for detecting bacteria comprising a polynucleotide primers and a probe consisting of the sequence

| | | |
|---|---|---|
| Seq ID No 3 | AGAGTTTGATCATGGCTCAGA | [primer forward] |
| Seq ID No 4 | TTACCCCACCTACTAGCTAAT | [primer reverse] |
| Seq ID No 12 | GAGTGGCGGACGGGTGAGTAA | [probe forward] |

5. The method and kit of claim 1 and 2, wherein the composition for detecting bacteria comprising a polynucleotide primers and a probe consisting of the sequence

| | |
|---|---|
| Seq ID No 5 | |
| GYGGAGCATGTGGYTTAATTCG | [primer forward] |
| Seq ID No 6 | |
| TTGCGCTCGTTRCGGGACTT | [primer reverse] |
| Seq ID No 13 | |
| ACAGGTGGTGCATGGTTGTC | [probe forward] |
| Seq ID No 14 | |
| TCAGCTCGTGTCGTGAGATGTT | [probe forward] |
| Seq ID No 15 | |
| ACAGGTGCTGCATGGCTGTC | [probe forward] |
| Seq ID No 16 | |
| TCAGCTCGTGTTGTGAAATGTT | [probe forward] |

6. The method and kit of claim 1 and 2, wherein the composition for detecting fungus-yeast comprising a polynucleotide primers and a probe consisting of the sequence

| | |
|---|---|
| Seq ID No 7 | |
| GGGAAACTCACCAGGTCCA | [primer forward] |
| Seq ID No 8 | |
| CGTTATCGCAATTAAGCAGACA | [primer reverse] |
| Seq ID No 17 | |
| AGGATTGACAGATTGAGAGCTCTT | [probe forward] |

7. The method and kit of claim 1 and 2, wherein the composition for detecting fungus-yeast comprising a polynucleotide primers and a probe consisting of the sequence

| | | |
|---|---|---|
| Seq ID No 9 | GGTAACGGGGAATWAGGGTTC | [primer forward] |
| Seq ID No 10 | TTGGGTAATTTGCGCGCCTG | [primer reverse] |
| Seq ID No 18 | CGGAGAGGGAGCCTGAGAA | [probe forward] |
| Seq ID No 19 | CGGCTACCACATCCAAGGAA | [probe forward] |

8. The method and kit of one of claims 1 to 6, wherein the preferred combination of primers and probes used for detection all bacteria and/or fungus-yeast consisting of the sequence:
Seq ID No 1+Seq ID No 2+Seq ID No 11
or
Seq ID No 3+Seq ID No 4+Seq ID No 12
or
Seq ID No 5+Seq ID No 6+Seq ID No 13+Seq ID No 14+Seq ID No 15+Seq ID No 16
or
Seq ID No 7+Seq ID No 8+Seq ID No 17
or
Seq ID No 9+Seq ID No 10+Seq ID No 18+Seq ID No 19
or
Seq ID No 1+Seq ID No 2+Seq ID No 11+Seq ID No 7+Seq ID No 8+Seq ID No 17
or
Seq ID No 3+Seq ID No 4+Seq ID No 12+Seq ID No 7+Seq ID No 8+Seq ID No 17
or
Seq ID No 5+Seq ID No 6+Seq ID No 13+Seq ID No 14+Seq ID No 15+Seq ID No 16+Seq ID No 9+Seq ID No 10+Seq ID No 18+Seq ID No 19

9. The method and kit of one of claims 1 to 8, wherein the polynucleotide primers and probes are natural nucleic acid or Peptide Nucleic Acid (PNA) which can hybridize to nucleic acid (DNA and RNA).

10. The method and kit of one of claims 1 to 9, and also quantified this RNA for a comparison with quantified external standard RNA from by exemple Escherichia coli and Candida spp.


SEQUENCE LISTING (19 sequences)

SEQ ID NO: 1 - DNA, 22 bp, Artificial
  Source (1)..(22): /note = “Description of artificial sequence oligonucleotide primer”
tggagcatgtggtttaattcga

SEQ ID NO: 2 - DNA, 19 bp, Artificial
  source (1)..(19): /note = “Description of artificial sequence oligonucleotide primer”
tgcgggacttaacccaaca

SEQ ID NO: 3 - DNA, 21 bp, Artificial
  source (1)..(21): /note = “Description of artificial sequence oligonucleotide primer”
agagtttgatcatggctcaga

SEQ ID NO: 4 - DNA, 21 bp, Artificial
  source (1)..(21): /note = “Description of artificial sequence oligonucleotide primer”
ttaccccacctactagctaat

SEQ ID NO: 5 - DNA, 22 bp, Artificial
  source (1)..(22): /note = “Description of artificial sequence oligonucleotide primer”
gyggagcatgtggyttaattcg

SEQ ID NO: 6 - DNA, 20 bp, Artificial
  source (1)..(20): /note = “Description of artificial sequence oligonucleotide primer”
ttgcgctcgttrcgggactt

SEQ ID NO: 7 - DNA, 19 bp, Artificial
  source (1)..(19): /note = “Description of artificial sequence oligonucleotide primer”
gggaaactcaccaggtcca

SEQ ID NO: 8 - DNA, 22 bp, Artificial
  source (1)..(22): /note = “Description of artificial sequence oligonucleotide primer”
cgttatcgcaattaagcagaca

SEQ ID NO: 9 - DNA, 21 bp, Artificial
  source (1)..(21): /note = “Description of artificial sequence oligonucleotide primer”
ggtaacggggaatwagggttc

SEQ ID NO: 10 - DNA, 20 bp, Artificial
  source (1)..(20): /note = “Description of artificial sequence oligonucleotide primer”
ttgggtaatttgcgcgcctg

SEQ ID NO: 11 - DNA, 23 bp, Artificial
  source (1)..(23): /note = “Description of artificial sequence oligonucleotide primer”
tgcatggytgtcgtcagctcgtg

SEQ ID NO: 12 - DNA, 21 bp, Artificial
  source (1)..(21): /note = “Description of artificial sequence oligonucleotide primer”
gagtggcggacgggtgagtaa

SEQ ID NO: 13 - DNA, 20 bp, Artificial
  source (1)..(20): /note = “Description of artificial sequence oligonucleotide primer”
acaggtggtgcatggttgtc

SEQ ID NO: 14 - DNA, 22 bp, Artificial
  source (1)..(22): /note = “Description of artificial sequence oligonucleotide primer”
tcagctcgtgtcgtgagatgtt

SEQ ID NO: 15 - DNA, 20 bp, Artificial
  source (1)..(20): /note = “Description of artificial sequence oligonucleotide primer”
acaggtgctgcatggctgtc

SEQ ID NO: 16 - DNA, 22 bp, Artificial
  source (1)..(22): /note = “Description of artificial sequence oligonucleotide primer”
tcagctcgtgttgtgaaatgtt

SEQ ID NO: 17 - DNA, 24 bp, Artificial
  source (1)..(24): /note = “Description of artificial sequence oligonucleotide primer”
aggattgacagattgagagctctt

SEQ ID NO: 18 - DNA, 19 bp, Artificial
  source (1)..(19): /note = “Description of artificial sequence oligonucleotide primer”
cggagagggagcctgagaa

SEQ ID NO: 19 - DNA, 20 bp, Artificial
  source (1)..(20): /note = “Description of artificial sequence oligonucleotide primer”
cggctaccacatccaaggaa